(19) United States
(12) Patent Application Publication
(10) Pub. No.: US 2022/0358647 A1
(43) Pub. Date: Nov. 10, 2022

(54) DEVICE AND METHOD FOR ATTESTING THE OPERATIONS OF AN IN-VITRO DIAGNOSTIC DEVICE

(71) Applicant: MACS S.R.L., Rome (IT)

(72) Inventors: Andrea EUGENI, Rome (IT); Marco CASTELLO, Genova (IT)

(21) Appl. No.: 17/777,064

(22) PCT Filed: Nov. 13, 2020
(86) PCT No.: PCT/IB2020/060684

§ 371 Date: May 16, 2022

(30) Foreign Application Priority Data
Nov. 15, 2019 (IT) 102019000021264

(51) Int. Cl. G06T 7/00; G06T 7/62; G06T 7/70; G06V 20/50; G01N 35/00; G01N 35/10; G16H 50/20; G16H 30/40

(52) U.S. Cl. CPC G06T 7/0012; G06T 7/62; G06T 7/70; G06V 20/50; G01N 35/00623; G01N 35/00722; G01N 35/00732; G01N 35/1016; G01N 35/1011; G16H 50/20; G16H 30/40; G06V 2201/034; G06V 10/40

(57) ABSTRACT

Device (100) for attesting the operations of an in-vitro diagnostic device (50) comprising: a block (101) for capturing a plurality of frames of the tip (51); a block (102) for storing the plurality of frames; a block (103) for evaluating the right hooking of the tip (51) to the in-vitro diagnostic device (50); a block (104) for evaluating the volume of a liquid contained in the tip (51); a block (105) for carrying out verification before the operation of dispensing the liquid; a block (106) for carrying out a post-dispensing verification; blocks (107, 108) for emitting electronic signals; a block (109) for integrating a system for managing the errors; a block (110) for saving the data; a block (111) for communicating with the in-vitro diagnostic device (50).

The present invention relates to a device for attesting the operations of an in-vitro diagnostic device.

The present invention also relates to a method for attesting the operations of an in-vitro diagnostic device.

In particular, the present invention relates to a device and a method for attesting the operations of an in-vitro diagnostic device of the type applicable to devices intended for aspiration and dispensing of fluids.

In the diagnostic field, different diagnostic systems useful to perform the analysis in vitro, and systems and methods useful to verify the correct operation of the diagnostic devices are known.

A first example is the patent US2016291049A1, which describes methods to determine or verify the proper functioning of a diagnostic device. Methods include monitoring, with one or more imaging devices, of one or more locations to determine aspiration, dispensing, and/or volume filling of the overall dosage components of one or more containers. The depth and dive location of a tip can also be measured and/or verified.

The patent application JP2003092749A, instead, describes a management system for managing the experiments in the laboratory. The system comprises a camera that detects a state associated with an experiment, a data collection device associated with the state of the experiment, and a transmitter that transmits the image collected by the camera and the collected data to a remote monitoring terminal via the Internet network. The data collection device uses a decision section, a signal generation section that generates a trigger signal when the decision section detects the change and transmits the result to the monitor terminal in real-time.

However, the systems like those here described suffer of the problem of using a camera that images the tube in which the liquid will be deposited and does not image instead of the tip, whereby the measure is an indirect measure. In fact, the aspirated volume is measured as the difference between the volume previously present and the volume present after aspiration. The dispensed volume is measured similarly to the difference between volumes. Therefore, it's not possible to reach sensitivity and resolution equal to microliter, often necessary for an effective and reliable solution. In these cases, it is also necessary to perform a perfect characterization of the container (test tube, in the sample case, or bottle, in the case of reagent) from which the liquid is aspirated or in which the liquid is deposited.

Instead, it would be much simpler and cheaper to characterize only the tip. Furthermore, the systems and methods of the known art do not certify the diagnostic result relative to the field of laboratory analysis.

The purpose of the present invention is to provide a device and a method for attesting the operations of an in-vitro diagnostic device simple and economical and which provides the certification of the diagnostic result, having, therefore, features such to overcome the limitations of existing devices and methods of certifying the operations of a diagnostic analysis machine.

According to the present invention, a device for attesting the operations of an in-vitro diagnostic device is provided, as defined in claim 1. According to the present invention also a method for attesting the operations of an in-vitro diagnostic device is provided, as defined in claim 7.

For a better understanding of the present invention, a preferred embodiment is now described, purely by way of non-limiting example, with reference to the attached drawings, in which:

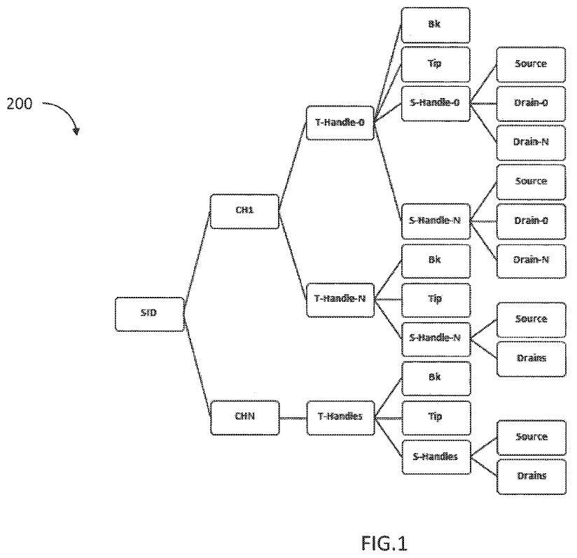

FIG. 1 shows a schematic view of a method for storing frames of a method for attesting the operations of an in-vitro diagnostic device, according to the invention;

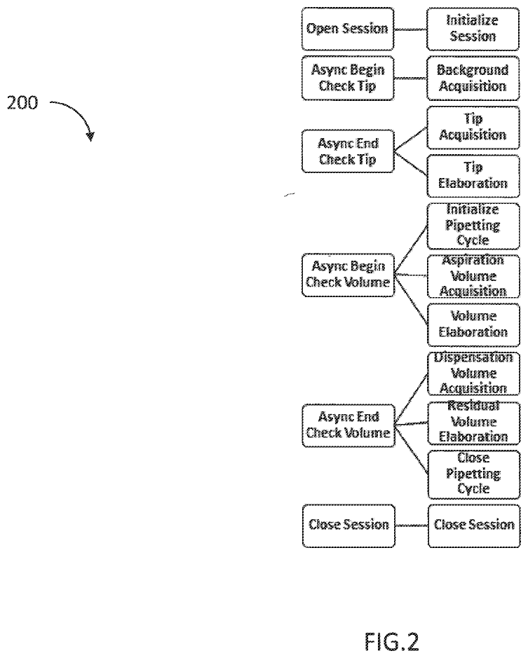

FIG. 2 shows a block diagram of the method for attesting the operations of an in-vitro diagnostic device, according to the invention;

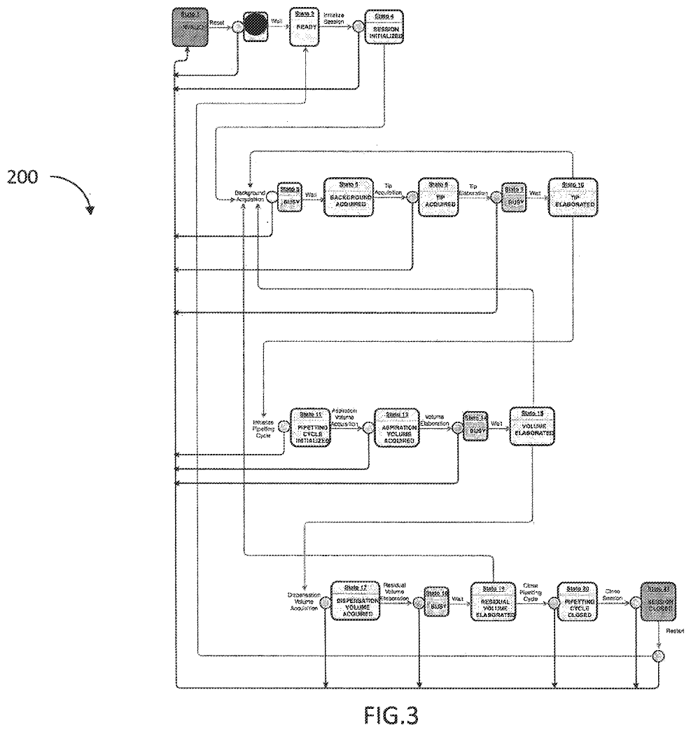

FIG. 3 shows a detailed block diagram of the method for attesting the operations of an in-vitro diagnostic device, according to the invention;

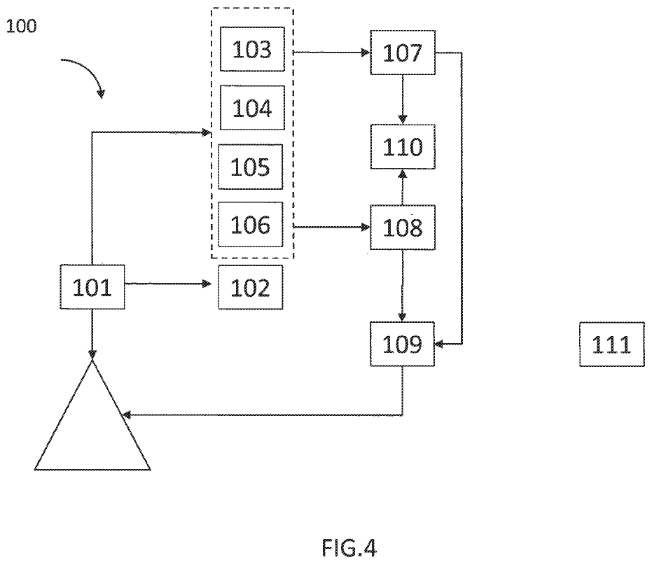

FIG. 4 shows a block diagram of a device for attesting the operations of an in-vitro diagnostic device, according to the invention;

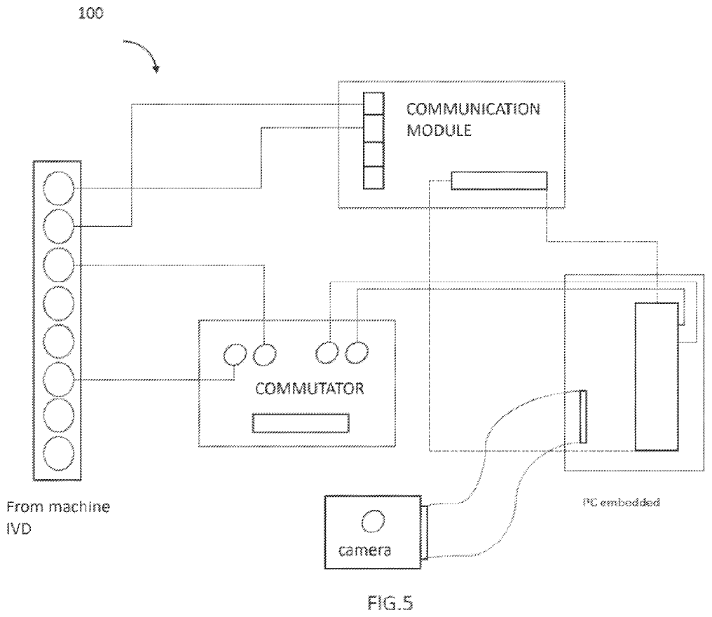

FIG. 5 shows a schematic view of the device for attesting the operations of an in-vitro diagnostic device, according to the invention;

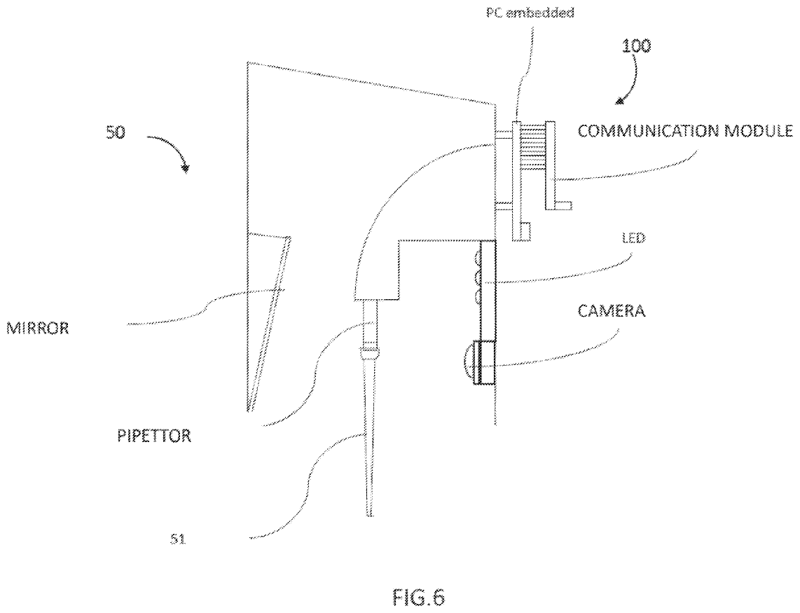

FIG. 6 shows a schematic view of the device for attesting the operations of an in-vitro diagnostic device, according to the invention.

With reference to FIG. 5, a device for attesting the operations of an in-vitro diagnostic device is shown.

The device 100 comprises:

a block 101 for capturing a plurality of frames of the tip 51 of an in-vitro diagnostic device 50 (IDV) to which the device 100 is attached or integrated;

a block 102 for storing the plurality of frames captured by the block 101 divided on the base of the work session, the channel of aspiration and pipetting and pipetting cycle to which said frames refer, to allow eventual future consultation;

a block 103 for evaluating the right hooking of the tip 51 to the in-vitro diagnostic device 50, in terms of presence, cleaning, size, and position (angle and grip) by means of a block for checking the cleaning, a block for verification of the size, the angle, and grip of the tip 51;

a block 104 for estimating the volume of liquid present in the tip 51 in terms of tip presence, presence of liquid, an estimate of the volume and anomalies of the liquid inside the tip 51, such as for the presence of bubbles or foam or the presence of particulate matter, fibrin, other foreign bodies, by means of verification of the presence block of the tip 51, a block of verification of the presence of liquid in the tip 51, a block for estimating the volume and anomalies of the liquid inside the tip 51;

a block 105 for the execution of an initial verification of the dispensation of the liquid contained in the tip 51, which includes a block for the verification of the expected volume presence and a block for the verification of the presence of the tip 51;

a block 106 for carrying out a post-dispensing verification comprising a block for verifying the presence of the tip 51 and a block for evaluating the volume of the dispensed liquid;

a block 107 for emitting an electronic signal able to signal the validation of the operation of drawing the liquid;

a block 108 for emitting an electronic signal able to signal the validation of the operation of dispensing the liquid;

a block 109 for integrating a system for managing the errors by means of a software program for controlling the in-vitro diagnostic device 50;

a block 110 for saving the data related to the certification of the operations concerning a single work session;

a block 111 for communicating with the in-vitro diagnostic device 50, for managing the device 100 by means of a communication channel to be chosen among RS485, RS232, CAN, Wifi, Ethernet, ZigBee, Bluetooth or Bluetooth Low-Energy.

In addition, according to an aspect of the invention, the device 100 comprises a camera for the acquisition of images of the tip 51 of the diagnostic device 50 in correspondence of the following phases: grip of the tip 51 corresponding to a time to, aspiration of the serum or reagent corresponding to a time t₁>t₀, dispensation of the serum or of the reagent corresponding to a time t₂>t₁ and release of the tip 51 corresponding to a time t₃>t₂.

According to an aspect of the invention, the device comprises a single camera for carrying out the above-mentioned operations.

According to another aspect of the invention, the device comprises a second camera configured to record the entire work session, store the video, and constitute the “black box” of the diagnostics device.

According to another aspect of the invention, the device 100 comprises a third camera oriented towards a work surface of the machine 50 IDV having the task of certifying the positioning of the machine precisely above the wells into which it is to dispense or over the bottles and tubes from which it has to aspirate and on the tips it has to hook.

According to an aspect of the invention, the third video camera is adapted to provide the IDV machine with information and indications such as to guide the exact positioning over the wells.

According to an aspect of the invention, the processing of the images acquired by the block 101 for acquiring a plurality of frames of a tip 51 can be performed by means of classification algorithms, artificial intelligence, and data codification, which include stages of:
acquiring a plurality of frames of a tip 51 by means of the acquisition block 101;
insert a digital signature of the plurality of frames;
checking the grip of the tip 51 by the machine 50;
check the dimensions of the tip 51;
perform a check of the position and grip angle of the tip 51;
check for any loss of the tip 51;
validate the suction volume of the tip 51;
check that the liquid contained in tip 51 has been dispensed;
check for clots;
check for the presence of foam.

In use, the images acquired during the working session of the in vitro diagnostic analysis machine 50 are stored along with relevant information (in particular: the date and time, the ID of the device, the ID of the instrument) and related information log about the operations and commands performed by the machine.

In case of a problem encountered during a session or an error signal, the images can be consulted and will provide valuable information to understand what happened during the analysis operations to identify the cause of the problem. The acquisition of frames, during all the most delicate phases of a work session, with a code ID and a digital signature, make them uniquely linked to the session and the specific action that the machine was running to be useful to verify that the whole procedure has been carried out correctly.

These frames are stored in the device 100 and can be easily consulted by an operator, but the digital signature makes them impossible to alter.

The device 100 acquires a frame when the machine 50 has taken the tip 51; checks that the tip 51 has been taken, and the information is classified and stored. In case the verification gives a negative result, or if the diagnostic machinery has not tried to take the tip, the device 100 will emit a first error signal adapted to inform the operator of the absence of the tip.

During the step of verifying the dimensions of the tip 51, the device 100 also checks whether the tip 51 is in accordance with the analysis protocol being carried out. If the verification gives a positive result, that is, if the tip is compliant, the device 100 classifies the information and stores it. In the event the taken tip was not the one expected, the device 100 emits a second error signal, adapted to inform the operator on the erroneous operation that the machine has performed.

In addition to the previous operations, the device 100 is able to check whether the position and the gripping angle of the tip are the optimal ones. Method 200 comprises the step of verifying the correct positioning and the gripping angle of the tip 51. In the event this verification gives a positive result, or in the case in which this information reflects the pre-set standards in the device 100, then the information is classified and stored. Otherwise, the device emits a third error signal, adapted to inform the operator that the tip of the gripping position is not correct and will be required to run the operation again.

In the step of verifying the leakage of the tip 51, at each acquisition the presence of the tip 51 mounted on the pipetting channel of the machine 50 is checked. If that verification fails, i.e. if the absence of the tip is verified, the device emits a fourth error signal and the operator is informed of the erroneous operation that the machine diagnostics performed.

In the phase of validating the volume of suction of the tip 51, the device 100 acquires an image of the tip 51 after performing operations for the suction of the liquid from a source, processes the frame, and provides indications on the volume of liquid contained in the tip. If that verification fails, namely in the case that the volume present in the tip 51 does not correspond to the volume necessary to carry out the dispensing operations, the device 100 emits a fifth error signal by means of which the operator is informed of the wrong operation that the machine has performed.

In the phase of verifying the occurred dispensing, the device 100 acquires an image of the tip 51 after the operations for dispensing the liquid in the wells is performed, processes the frame, and provides indications on the volume of the liquid contained in the tip. If this fails to occur, or in case it is found that the volume present in the tip does not correspond to the expected residual volume, the device 100 emits a sixth error signal by which the operator is informed of the erroneous operation that the machine ran.

The phase of checking for the presence of clots is carried out since the presence of clots can plug the slot in the tip and make the dispensing ineffective. Artificial intelligence systems and image analysis make it possible to analyze the integrity and homogeneity of the aspirated sample or reagent. If this verification fails, or in case the presence of clots inside the aspirated liquid would be identified, the device 100 emits a seventh error signal by means of which the operator is informed of the erroneous operation that the machine has performed.

The phase of the identification of foam or bubbles is carried out because the presence of foam in the reagents can fool the system for searching the level of the machine leading to inconsistent volumes aspirations. Artificial intelligence and image analysis systems make possible the analysis of the homogeneity of the aspirated liquid. If this verification fails, or the presence of foam or air bubbles inside the liquid aspirated has been identified, the device 100 emits an eighth error signal by means of which the operator is informed of the erroneous operation that the machine ran.

According to an aspect of the invention, one or more of the first, second, third, fourth, fifth, sixth, seventh, and eighth error signals are light signals emitted by the device 100.

According to an aspect of the invention, one or more of the first, second, third, fourth, fifth, sixth, seventh, and eighth error signals are sound signals emitted by the device 100.

According to an aspect of the invention, the device 100 is powered by a power supply switching capable of managing supply voltages between 48V and 5V.

According to an aspect of the invention, the device 100 is compact and can be integrated into known diagnostic machines.

According to an aspect of the invention, the device 100 includes a camera having a resolution higher than or equal to 5 Megapixels.

With reference to these figures and, in particular, to FIGS. 2 and 3, it is provided a method 200 for attesting the operations of an in-vitro diagnostic device comprising the steps of:
validating the volume of suction;
validating the aspiration volume;
checking the clots;
checking the foam;
acquiring a plurality of frames;
digitally signing digital a plurality of frames;
checking the grip of the tip 51;
checking the size of the tip 51;
checking the position and the gripping angle of the tip 51;
identifying the loss of the tip 51.

In addition, the method 200 comprises the step of communicating with a system for controlling the machine 50, which comprises the steps of:
receiving commands for the configuration of the device 100;
receiving commands for the activation of functions;
receiving commands to request the frames acquired;
receiving commands for the status request;
receiving commands for the request for interpretation of the frames;
managing the acquisition of the frame;
certifying the frame;
performing the operations of analyzing the image (pretreatment, segmentation, etc.) to allow the subsequent activity of the pattern in the identification and estimation;
identifying the presence of tip, of liquid, the volume present at the edge of the tip;
performing estimates on the following physical parameters: Length of the tip; present volume on the tip.

With reference to FIG. 1, in the block diagram of method 200, the acronyms shown have the following meanings:

SID=session ID: unique identification key of the analysis session;

CH1 . . . CHN=channel1 . . . channel n°: indicates the pipetting channel, each machine 50 can have from 1 to n° channels;

T-Handle-0 . . . T-Handle-N=Tip Handle0 . . . Tip Handle n°: indicates the unique identifier of the tip object 51, with each pipetting channel it is possible to take from 0 to n° tips 51;

Bk=background: image taken before the step of hooking the tip 51;

Tip=tip: image taken immediately after gripping the tip 51;

S-Handle-0 . . . S-HandleN=source handle 0 . . . source handle n°: indicates the identification of the bottle from which the liquid is sucked, for each channel and for each tip 51 they can be from 0 to n°;

Source=image taken immediately after having aspirated the liquid from the source bottle;

Drain0 . . . DrainN=images taken immediately after dispensing the liquid into the well. The liquid aspirated with a tip 51 can be dispensed on 0-n° different wells.

FIG. 3 shows a detail of the method 200 according to the invention.

In particular, in the phase of storing the frames for the certification of the work session, each session is uniquely identified by an ID. Within each session, the work performed by each of the N possible pipetting channels is divided. Each pipetting channel can hook one tip at a time, uniquely identified by an ID. This identifier will accompany the entire life cycle of the tip 51, from the moment of gripping to that of release. For each tip 51 an image will be acquired before and another after the gripping operation, which will be used for the classification analysis of the presence, type, and conformity of the tip 51. Each tip 51 can draw liquid from a single test tube, called source, which is also univocally identified by an ID and can perform one or more dispensations to one or more taps on different wells, called drains. The pipetting cycle is defined as the operations carried out between one aspiration and the next. For each pipetting cycle, an image will be acquired at the end of aspiration and processed to verify the correctness of the quantity and homogeneity of the aspirated liquid and an image at the end of each dispensing to verify that the correct volume of liquid has been dispensed. At the end of the session, the folder is encrypted and archived for future consultation and checks. In the states from “Invalid” to “Ready”, it is checked that the device 100 from an initial state is able to complete all the checks of its functionality before receiving the first command. This group of commands is synchronized with the switching on or the reset/restart of the machine 50 hosting it so that the latter does not have to wait for the device 100 to be in the Ready position before starting to operate. Once this state is reached, it will be synchronized with machine 50. The second group of admissible states ranging from state 4: Session Initialized up to Tip Elaborated are those related to the control of the tips. The device, to the state 6 has checked that pipettors had not still hooked the tips of the previous cycle and of having carried out a correct acquisition of the pipettors' background on which it is going to connect with the new tips. Once the pipettor or pipettors (based on the number of channels that the IVD machinery supports) has taken the tips, the device will acquire this information and will go to process if the tip 51 that has been equipped is the one that is correctly foreseen by the current phase of the test method in progress. If the tip 51 is the correct one, it is possible to pass to the successive states which go from state 11 to state 15 of the processing of the aspirated volume. These are used to check if the volume in the tips is consistent with that set in the test method, if we are in the presence of bubbles, if there is particulate that could obstruct correct dispensing, etc. Once the volume check has been performed, and the device has detected that the volume is the correct one, it moves to states 17 to 20 where the volume is dispensed and the device performs an analysis on the volume remaining in the tips. If this processing has also not detected any anomalies, then the last state allowed by the machine before starting the cycle again is Session Closed. At any moment, it is not possible to reach one of the admissible states, an error is generated and the system returns to the Invalid state before being able to restart.

The device 100 communicates at the hardware level with the machine 50 IVD through a communication protocol RS485, or RS232, ZigBee, CAN. The implemented commands and the relative parameters that are passed as additional information to perform the specific operations and processing are the following: Session ID, Source Handle Iteration, Channels Pattern, Tip Handle, Expected Volume, Detected Volume. This information is exchanged between the device 100 and the machine 50.

The method 200 is based on artificial vision and intelligence systems to certify the work session of the in vitro diagnostic analysis machine 50 to which it is applied.

The images are acquired, then a phase of pre-processing follows, feature extraction, and finally classification through machine learning algorithms, to create an approximate model of the three-dimensional real world starting from two-dimensional images.

Advantageously, the device and the method according to the present invention reproduce the checks carried out from the human sight of the laboratory technician not only with the acquisition of a two-dimensional image, but above all with the interpretation of the content of that image. The information that is extracted implies an automatic decision by the machine, which will validate or not the operation performed by the machine. Being an artificial vision system, it consists of the integration of optical, electronic, and mechanical components that allow to acquire, record, and process images in both the visible and infrared light spectrum. The result of the processing is the recognition of certain characteristics of the image for the purpose of checking tip grip and volume, tip type classification, selection of the consistency of the liquid, etc. The frame collected from the image is stored in a system of encrypted folders or an encrypted database; the data relating to the entire session are attached to the log file of the same. The same frame is processed in order to perform the tip analysis.

There is a typical pre-processing phase consisting of the following operations:—transformation of the image into grayscale;—segmentation of the image around the set ROIs (regions of interest);—noise filtering (denoising) of the image to reduce noise;—bilateral Gaussian filtering to make the image more uniform, without altering the contrast of the edges of the figures. This preliminary processing phase is followed by a specific processing and extraction phase of the features based on the purpose of the processing being carried out. Specifically, as regards the recognition of presence and absence and tip typology, the aim is to extract features that characterize the presence of an object in the image and its dimensions, deriving from the comparison with the image taken immediately before the phase of tip grip that is used as a background image. Some of the metrics referred to are: result of a mathematical comparison between the image and the background;—index of structural similarity between the image and the background;—mean square error in the comparison between the image and the background;—perimeter, intended as the number of pixels that make up the edge of the object identified with the object detection algorithm in the image. As for the recognition of the correctness of the tip grip, the aim is to extract features that characterize the geometric structure of the objects present in the image. The metrics referred to are:—Edge points extremal, the value of the edge point measured further down;—SxSlope and DxSlope, value of the slope of the straight line obtained from the regression of the edge points detected in the left and right half of the image. As for the volume level of aspirated and dispensed liquid, the aim is to extract features that highlight the distance between the top of the tip and the level of separation between liquid and air in the tip. Since the geometry of the tip 51 is known after having classified its type, it is possible to associate the volume with the measurement of the vertical distance between the two points. The metrics referred to are:—segmentation of the point image only by comparison between image and background, thresholding and application of a mask;—application of the same mask to the image of the tip containing a volume of liquid;—template matching with a model of the tip and template matching with a model of a level;—identification of the presence of lines and horizontals through Hough Transform Algorithm or another transform;—count of the subtraction pixels between the image of the empty tip and the tip full of liquid.

A phase of learning of the classification algorithm always precedes the phase of use of the classifier itself.

To this end, acquisition sessions of a set of data are prepared for which each image is accompanied by the label of the class corresponding to it and are used as input data to train the classifier. The VitroCERT system adds an online training phase. The invention comprises an interface that shows the scanned image and the prediction that the software has executed to the laboratory technician. Advantageously, a technician has the possibility to validate or, in the event of a classifier error, correct the prediction. The securely labeled image will be added to the training database and will increase the accuracy and precision of the classification system. A different classifier is used for each classification objective: Convolutional Neural Network, Decision Tree, Perceptron, Artificial Neural Network.

Thus, the device and the method for attesting the operations of an in-vitro diagnostic device according to the invention consent to verify and certify the correctness of all the operations relating to in vitro analysis.

Another advantage of the device and method for attesting the operations of an in-vitro diagnostic device according to the invention consists in the fact of providing a signal to alert the operator in case one or more operations and relative parameters are not in compliance and correct.

Finally, the device and method for attesting the operations of an in-vitro diagnostic device according to the invention are economical.

Finally, it follows that the device and the method for attesting the operations of an in-vitro diagnostic device here described and illustrated may be subject to modifications and variations without thereby departing from the scope of the present invention as defined in the attached claim.

1. Device for attesting the operations of an in-vitro diagnostic device, having a work session comprising the operations of hooking at least a tip, drawing and dispensing a liquid, comprising: a block for capturing a plurality of frames of the tip of the in-vitro diagnostic device to which said device is fixed; a block for storing the plurality of frames captured by the block divided on the base of the work session, a channel of aspiration and pipetting, and a pipetting cycle to which said frames refer; a block for evaluating the right hooking of the tip to the in-vitro diagnostic device;—a block for evaluating the volume of a liquid contained in the tip; a block for carrying out verification before the operation of dispensing the liquid contained in the tip, comprising a block for verifying the expected volume and a block for verifying the presence of the tip; a block for carrying out a post-dispensing verification comprising a block for verifying the presence of the tip and a block for evaluating the volume of the dispensed liquid; a block for emitting an electronic signal able to signal the validation of the operation of drawing the liquid; a block for emitting an electronic signal able to signal the validation of the operation of dispensing the liquid; a block for integrating a system for managing the errors by means of a software program for controlling the in-vitro diagnostic device;—a block for saving the data related to the certification of the operations concerning a single work session; a block for communicating with the in-vitro diagnostic device, for managing the device by means of a communication channel; characterized in that in that said block comprises a block for verifying the presence of the tip, a block for verifying the cleaning of the tip, a block for verifying the dimensions, the angle, and the picking up of the tip.

2. Device for attesting the operations of an in-vitro diagnostic device according to claim 1 characterized in that said block for capturing a plurality of frames comprises at least a camera able to capture frames at prefixed instants: picking up the tip at a time to, drawing the serum or reagent, at a time t₁>t₀, dispensing the serum or the reagent at a time t₂>t₁ and dropping the tip at a time t₃>t₂.

3. Device for attesting the operations of an in-vitro diagnostic device according to claim 1 characterized in that said block for capturing a plurality of frames comprises at least a second camera able to shoot an entire work session and to save the footage.

4. Device for attesting the operations of an in-vitro diagnostic device according to claim 1 characterized in that said block for evaluating the volume of the liquid contained in the tip comprises a block for verifying the presence of the tip, a block for verifying the presence of the liquid into the tip, a block for evaluating the volume and anomalies of the liquid into the tip.

5. Device for attesting the operations of an in-vitro diagnostic device according to claim 1 characterized in comprising an interface provided with a display able to show the captured frames of the tip and expected numerical values of the device, and means for emitting optical od acoustic warning signals.

6. Method for attesting the operations of an in-vitro diagnostic device by means of the device according to claim 1, comprising the steps of:
capturing a plurality of frames of a tip through the block;
affixing a digital signature of the plurality of frames;
verifying the picking up of the tip made by the in-vitro diagnostic device and emitting a first warning signal in case the picking up is not compliant;
verifying the dimensions of the tip and emitting a second warning signal in case the tip is not compliant, classifying and storing the data on the contrary;
verifying the correct positioning and the angle of the picking up of the tip and emitting a third warning signal in case of position or angle of picking up not compliant with values prefixed on the device;
verifying the possible slipping of the tip and emitting a fourth warning signal in case of slipping of the tip;
validating the volume of aspiration of the tip on the base of the processing of a frame captured by the device after an operation of liquid drawing, and emitting a fifth warning signal in case the volume doesn't match a prefixed value;
verifying the occurrence of dispensing the liquid contained in the tip on the base of the processing of a frame captured by the device after an operation of dispensing the liquid contained in the tip, and emitting a sixth warning signal in case the volume of the liquid detected into the tip is not compliant with a prefixed value;
verifying the presence of clots by means of artificial intelligence systems and image analysis, and emitting a seventh warning signal in case a clot is detected;
verifying the presence of foam by means of artificial intelligence systems and image analysis, and emitting an eighth warning signal in case foam is detected;
one or more within the first, the second, the third, the fourth, the fifth, the sixth, the seventh, and the eighth warning signal are optical signals emitted by the device;
one or more within the first, the second, the third, the fourth, the fifth, the sixth, the seventh, and the eighth warning signal are sound signals emitted by the device;

7. Method for attesting the operations of an in-vitro diagnostic device according to claim 6 characterized in comprising a step of storing a plurality of frames captured together with date and time related to the capture, a device ID, a ID of the instrument related to the operation.

8. Method for attesting the operations of an in-vitro diagnostic device according to claim 6 characterized in comprising a step of communicating with an instrument control system comprising:
receiving commands for the configuration of the device;
receiving commands for activating functions;
receiving commands for the request of the captured frames;
receiving commands for the request of the status;
receiving commands for the request of interpretation of the frames;
managing the capture of the frame;—validating the frame;
carrying out the operations of image analysis to allow the subsequent activities of identification of pattern and evaluation;—identifying the presence of the tip, the presence of liquid, of foam or bubbles; carrying out evaluations regarding the following physical parameters: length of the tip; volume present in the tip and sending to the device the related expected numerical values.

9. Method for attesting the operations of an in-vitro diagnostic device according to claim 6 characterized in comprising:
a step of preprocessing the plurality of frames captured by the block comprising: converting an image corresponding to a frame to grayscale; segmenting the image around the set Regions Of Interest (ROI); filtering the noise of the image to reduce the noise; gaussian bilateral filtering to make the image more uniform;
a step of processing and extracting specific features on the base of the step of verification in progress.
